(19) United States
(12) Patent Application Publication
(10) Pub. No.: US 2025/0223417 A1
(43) Pub. Date: Jul. 10, 2025

(54) WATER REMOVAL IN A PROCESS FOR HYDROLYTICALLY DEPOLYMERIZING A POLYAMIDE

(71) Applicant: BASF SE, Ludwigshafen am Rhein (DE)

(72) Inventors: Stefan BLEI, Ludwigshafen am Rhein (DE); Faissal-Ali EL-TOUFAILI, Ludwigshafen am Rhein (DE); Jochen GAUER, Ludwigshafen am Rhein (DE); Alexander HETZ-HUNSINGER, Ludwigshafen am Rhein (DE); Zeljko KOTANJAC, Ludwigshafen am Rhein (DE); Vikram Raghavendhar RAVIKUMAR, Ludwigshafen am Rhein (DE)

(21) Appl. No.: 18/852,637

(22) PCT Filed: Mar. 30, 2023
(86) PCT No.: PCT/EP2023/058270

§ 371 Date: Sep. 30, 2024

(30) Foreign Application Priority Data
Apr. 1, 2022 (EP) 22166281.0

(51) Int. Cl. C08J 11/14

(52) U.S. Cl. CPC C08J 11/14; C08J 2377/02

(57) ABSTRACT

The present invention relates to a water-efficient process for hydrolytically depolymerizing a polyamide prepared from ε-caprolactam.

The present invention relates to a water-efficient process for hydrolytically depolymerizing a polyamide prepared from ε-caprolactam.

Polyamide, and in particular polyamide 6 being characterized by the formula (—NH—(CH₂)₅—CO—)ₙ, can be found in numerous materials, such as packaging, engineering plastics from automotive and textile filaments. The latter represents about 40% of the polyamide 6 global market. At present, only a very small part of the textile filaments is recycled while it represents a significant percentage of the global CO₂ emissions. There is thus a need to recycle polyamide 6 from such materials. Further, the processes in the art are energy-intensive processes. Thus, there is a need to provide an improved process for depolymerizing a polyamide able to overcome these issues.

Surprisingly, it was found that the process of the present invention according to which polyamide is hydrolytically depolymerized is more robust and is more cost effective. Further, the process of the present invention permits to reduce the overall water consumption compared to known processes for depolymerization of polyamide 6, which reduction also permits to reduce costs. Hence, using a water-efficient process for depolymerizing a polyamide according to the present invention permits to reduce the CO₂ footprint.

Therefore, the present invention relates to a water-efficient process for hydrolytically depolymerizing a polyamide prepared from ε-caprolactam, said polyamide being comprised in a solid material M, the process comprising
(i) preparing an aqueous liquid stream S_{WC} containing ε-caprolactam dissolved in water, comprising
   (i.1) providing the solid material M containing the polyamide;
   (i.2) providing an aqueous liquid stream S_{W};
   (i.3) preparing an aqueous mixture of the solid material M provided according to (i.1) and the aqueous liquid stream S_{W} provided according to (i.2);
   (i.4) subjecting the aqueous mixture prepared according to (i.3) to depolymerization conditions in a chemical reactor unit R_{U}, obtaining the aqueous liquid stream S_{WC} containing ε-caprolactam dissolved in water;
(ii) separating water from the aqueous liquid stream S_{WC} by evaporation in at least two evaporation units, obtaining at least one aqueous vapor stream S_{V}, wherein at least a part of at least one aqueous vapor stream S_{V} is recycled into step (i.2) as a component of the aqueous liquid stream S_{W}.

Preferably, preparing an aqueous liquid stream S_{WC} containing ε-caprolactam dissolved in water according to (i) comprises
(i.1) providing the solid material M containing the polyamide, M having a temperature T_{M}, wherein T_{M}<T_{P}, T_{P} being the melting point of the polyamide;
(i.2) providing the aqueous liquid stream S_{W}, wherein from 50 weight-% to 100 weight-% of S_{W} consist of water and wherein S_{W} has a temperature T_{SW}, wherein T_{SW}>T_{P};
(i.3) preparing an aqueous mixture of the solid material M provided according to (i.1) and the aqueous liquid stream S_{W} provided according to (i.2), comprising feeding the solid material M provided according to (i.1) and the liquid aqueous stream S_{W} provided according to (i.2) into a chemical reactor unit R_{U} obtaining said mixture;
(i.4) subjecting the aqueous liquid mixture prepared according to (i.3) to depolymerization conditions in the chemical reactor unit R_{U}, obtaining the aqueous liquid stream S_{WC} containing ε-caprolactam dissolved in water, wherein the depolymerization conditions comprise a depolymerization temperature T_{D} at a depolymerization pressure p_{D}, wherein T_{M}<T_{D}<T_{SW}.

Preferably, T_{D} is in the range of from 230 to 320° C., more preferably in the range of from 250 to 300° C., more preferably in the range of from 250 to 295° C. or from 270 to 295° C.

Preferably, T_{SW} is in the range of from 250 to 350° C., more preferably in the range of from 260 to 330° C., more preferably in the range of from 290 to 325° C.

The excess heat from the liquid aqueous stream S_{W} (T_{SW}) melts the solid material M containing the polyamide in the chemical reactor unit R_{U}, wherein the solid material M is preferably in the form of granules, and provides the needed reaction enthalpy. Any additional heat required to maintain the reaction temperature can be provided through a heating jacket using hot oil of the reactor unit R_{U}. This is detailed in the following.

Preferably, ΔT=T_{SW}−T_{P} is in the range of from 10 to 70° C., more preferably in the range of from 10 to 50° C., more preferably in the range of from 10 to 30° C.

Preferably, p_{D} IS in the range of from 40 to 120 bar, more preferably in the range of from 50 to 100 bar, more preferably in the range of from 60 to 90 bar.

Preferably, in the mixture prepared according to (i.3), at least 75 weight-%, more preferably from 75 to 100 weight-%, more preferably from 85 to 100 weight-%, more preferably from 95 to 100 weight-%, of the polyamide comprised in the solid material M are comprised in liquid form.

Preferably, from 91 to 100 weight-%, more preferably from 92 to 100 weight-%, more preferably from 95 to 100 weight-% of S_{W} provided according to (i.2) consist of water.

Preferably, the solid material M provided according to (i.1) comprises, more preferably consists of waste material, wherein said waste material more preferably comprises textile waste material.

Preferably from 10 to 99 weight-%, more preferably from 30 to 98.5 weight-%, more preferably from 50 to 98 weight-%, more preferably from 80 to 98 weight-%, of M consist of the polyamide; or preferably from 10 to 100 weight-%, more preferably from 30 to 100 weight-%, more preferably from 50 to 100 weight-%, more preferably from 80 to 100 weight-%, of M consist of the polyamide.

Preferably, M is in the form of granules, wherein the mean diameter of the granules is more preferably in the range of from 0.5 to 10 mm, more preferably in the range of from 1 to 7 mm, more preferably in the range of from 2 to 4 mm.

According to (iii), M and S_{W} are preferably fed into R_{U} at a mixing ratio m_{W}/kg:m_{P}/kg, defined as the amount of water contained in S₁, m_{W}, relative to the mass of polyamide contained in M, m_{P}, in the range of from 1:1 to 20:1, more preferably in the range of from 2:1 to 15:1, more preferably in the range of from 5:1 to 10:1.

The mixing is preferably ensured by dosing the amount of water (S_{W}) stepwise with ongoing reaction time, and dosing the solid material M in the reactor unit R_{U}.

Preferably, M and S_{W} are fed into R_{U} one after the other. More preferably, S_{W} is fed into R_{U} and subsequently M is fed into R_{U} containing S_{W}.

Preferably, the chemical reactor unit R_{U} comprises z chemical reactors Rᵢ, i=1 . . . z, wherein z is in the range of from 1 to 10, more preferably in the range of from 2 to 8, more preferably in the range of from 2 to 6, more preferably in the range of from 2 to 5, more preferably in the range of from 2 to 4, more preferably 3 or 4, more preferably 4.

Preferably, z>1 and at least two reactors Rᵢ, more preferably the z reactors Rᵢ are serially coupled.

Preferably, the z reactors Rᵢ are serially coupled, wherein
M and S_{W} are fed into Rᵢ, i=1;
an aqueous liquid stream Sᵢ containing ε-caprolactam dissolved in water is removed from Rᵢ and fed into Rᵢ₊₁, i<z;
S_{WC} is removed from R_{z};
   wherein in every reactor Rᵢ, a depolymerization temperature T_{Di} at a depolymerization pressure p_{Di} are maintained, wherein, independently of each other, T_{Di} is in the range of from 230 to 320° C., more preferably in the range of from 250 to 300° C., more preferably in the range of from 270 to 295° C., and wherein, independently of each other, p_{Di} is more preferably in the range of from 40 to 120 bar, more preferably in the range of from 50 to 100 bar, more preferably in the range of from 60 to 90 bar.

Preferably maintaining a depolymerization temperature T_{Di} in a reactor Rᵢ comprises heating the reactor contents of Rᵢ, more preferably indirectly heating the reactor contents of Rᵢ, wherein more preferably, maintaining a depolymerization temperature T_{Di} in a reactor Rᵢ comprises heating the reactor contents of Rᵢ by passing a heating medium through a heating jacket of Rᵢ. The heating medium is preferably hot oil. However, it is noted that other heating medium known by the skilled person can be used for passing through the heating jacket of Rᵢ.

Preferably, the z reactors Rᵢ are vertically arranged, with R₁ being the top-most reactor and R_{z} being the bottom-most reactor, wherein Sᵢ obtained from Rᵢ is transferred to Rᵢ₊₁ by gravity, more preferably by gravity only.

Preferably, at least 1, more preferably z reactors Rᵢ, are configured as non-stirred reactors or as non-circulation reactors, more preferably as non-stirred reactors and non-circulation reactors.

The system is preferably operated without stirrer and without circulation pump. That avoids difficult sealings, i.e. high pressure sealings, for the stirrer and pump.

As mentioned in the foregoing, the mixing is preferably ensured by dosing the amount of water stepwise with ongoing reaction time, and dosing the liquid solution, for example, from reactor R₁ by gravity to reactor R₂ and R₃ after parts of the reaction time.

Preferably, the reactors R₁ to R_{y-1} are operated in batch mode and the reactors R_{y} to R_{z} are operated in continuous mode, wherein y>1 and y≤z, wherein y is more preferably z.

Preferably, the residence time in one or more of the reactors R₁ to R_{y-1}, more preferably in the reactors R₁ to R_{y-1}, is in the range of from 5 to 40 minutes, more preferably in the range of from 10 to 30 minutes, more preferably in the range of from 15 to 25 minutes.

Preferably, the residence time in the reactor R_{y}, wherein y is more preferably z, is in the range of from 1 second to 40 minutes, more preferably in the range of from 2 seconds to 30 minutes, more preferably in the range of from 3 seconds to 25 minutes.

Preferably, the overall residence time in the chemical reactor unit is in the range of from 15 to 160 minutes, more preferably in the range of from 30 to 120 minutes, more preferably in the range of from 45 to 100 minutes, more preferably in the range of from 60 to 80 minutes.

More preferably, four identical chemical reactors R₁-R₄ are arranged in series. The first three reactors, R₁-R₃, are preferably operated in batch mode, all of said reactors having low residence time and the last, R₄, in continuous mode to enable continuous feeding of the downstream process steps.

As to the feeding of S_{W} and M, it is preferred that M and S_{W} are fed into Rᵢ one after the other.

More preferably, S_{W} is fed into Rᵢ and subsequently M is fed into Rᵢ containing S_{W}. After a time T, Sᵢ is removed from Rᵢ and fed to Rᵢ₊₁.

Preferably, the aqueous liquid stream S_{WC} removed from R_{U} contains, in addition to water and ε-caprolactam, one or more chemical compounds dissolved in water and one or more compounds suspended in water. Generally, the composition of the aqueous liquid stream S_{WC} will depend on the composition of the solid material M (waste material).

Preferably, (ii) comprises
(ii.1) optionally feeding the aqueous liquid stream S_{WC} as a feed stream to a first evaporation unit EU1, obtaining at least one aqueous vapor stream S_{V1}, and an aqueous liquid stream S_{L1} comprising ε-caprolactam dissolved in water;
(ii.2) optionally feeding the aqueous liquid stream S_{WC}, or the aqueous liquid stream S_{L1}, to a solid-liquid separation unit, SLU, obtaining an aqueous liquid stream S_{SLU} comprising ε-caprolactam dissolved in water;
(ii.3) feeding the aqueous liquid stream S_{WC}, or the aqueous liquid stream S_{L1}, or the aqueous liquid stream S_{SLU}, to evaporation in at least two, more preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained;
   wherein more preferably from 75 to 100 weight-% of the aqueous liquid stream which is fed to evaporation according to (ii.3) consist of water and ε-caprolactam, said stream exhibiting a water concentration c_{H2O} and more preferably having a concentration of ε-caprolactam c_{CPL} in the range of from 5 to 20 weight-%;
   the process further comprising recycling at least a part of at least one of streams S_{V2} and S_{V3} into step (i.2) as a component of the aqueous liquid stream S_{W}, said recycling more preferably comprising condensing the at least one of streams S_{V2} and S_{V3}.

Preferably, the evaporation unit EU1 comprises, more preferably consists of, one or more flash drums.

Preferably, (ii) comprises
(ii.1) feeding the aqueous liquid stream S_{WC} as a feed stream to a first evaporation unit, EU1, obtaining at least one aqueous vapor stream S_{V1}, and an aqueous liquid stream S_{L1} comprising ε-caprolactam dissolved in water;
(ii.2) optionally feeding the aqueous liquid stream S_{L1} to a solid-liquid separation unit SLU, obtaining an aqueous liquid stream S_{SLU} comprising ε-caprolactam dissolved in water;
(ii.3) feeding the aqueous liquid stream S_{L1}, or the aqueous liquid stream S_{SLU}, to evaporation in at least two, more preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.

Preferably, according to (ii.1), the aqueous liquid stream S_{WC} is fed to evaporation by depressurization in EU1, obtaining the at least one aqueous vapor stream S_{V1} and the aqueous liquid stream S_{L1}. More preferably, p_{D} in R_{U} is in the range of from 40 to 120 bar, more preferably in the range of from 50 to 100 bar, more preferably in the range of from 60 to 90 bar and, according to (ii.1), the aqueous liquid stream S_{WC} is fed to evaporation by depressurization in EU1, obtaining the at least one aqueous vapor stream S_{V1} and the aqueous liquid stream S_{L1}.

Preferably, the process further comprises recycling at least a part of at least one aqueous vapor stream S_{V1} into step (i.2) as a component of the aqueous liquid stream S_{W}, wherein recycling at least a part of at least one stream S_{V1} into step (i.2) more preferably comprises condensing at least a part of at least one stream S_{V1}.

Preferably, the process comprises using at least a part of at least one aqueous vapor stream S_{V1} for providing heat to at least one heat-consuming unit in the water-efficient process for hydrolytically depolymerizing a polyamide prepared from ε-caprolactam. More preferably, the process further comprises recycling at least a part of at least one aqueous vapor stream S_{V1} into step (i.2) as a component of the aqueous liquid stream S_{W}, wherein recycling at least a part of at least one stream S_{V1} into step (i.2) more preferably comprises condensing at least a part of at least one stream S_{V1} and the process comprises using at least a part of at least one aqueous vapor stream S_{V1} for providing heat to at least one heat-consuming unit in the water-efficient process for hydrolytically depolymerizing a polyamide prepared from ε-caprolactam.

Preferably, (ii.1) comprises
(ii.1.1) feeding the aqueous liquid stream S_{WC} as a feed stream to a first evaporation sub-unit EU11, obtaining an aqueous vapor stream S_{V11}, and an aqueous liquid stream S_{L11} comprising ε-caprolactam dissolved in water;
(ii.1.2) feeding the aqueous liquid stream S_{L11} as a feed stream to a second evaporation sub-unit EU12, obtaining an aqueous vapor stream S_{V12}, and the aqueous liquid stream S_{L1} comprising ε-caprolactam dissolved in water. This is in particular illustrated in FIG. 2.

Preferably, according to (ii.1.1), the aqueous liquid stream S_{WC} is fed to evaporation by depressurization in EU11, obtaining the at least one aqueous vapor stream S_{V11} and the aqueous liquid stream S_{L11}, and wherein the aqueous liquid stream S_{L11} is fed to evaporation by depressurization in EU12, obtaining the at least one aqueous vapor stream S_{V12} and the aqueous liquid stream S_{L1}. More preferably, p_{D} in R_{U} is in the range of from 40 to 120 bar, more preferably in the range of from 50 to 100 bar, more preferably in the range of from 60 to 90 bar and, according to (ii.1.1), the aqueous liquid stream S_{WC} is fed to evaporation by depressurization in EU11, obtaining the at least one aqueous vapor stream S_{V11} and the aqueous liquid stream S_{L11}, and wherein the aqueous liquid stream S_{L11} is fed to evaporation by depressurization in EU12, obtaining the at least one aqueous vapor stream S_{V12} and the aqueous liquid stream S_{L1}.

Preferably, the process further comprises recycling at least a part of at least one of aqueous vapor streams S_{V11} and S_{V12} into step (i.2) as a component of the aqueous liquid stream S_{W}, wherein recycling at least a part of at least one of aqueous vapor streams S_{V11} and S_{V12} more preferably comprises condensing at least a part of at least one of streams S_{V11} and S_{V12}.

Preferably, the process comprises using at least a part of at least one of the aqueous vapor streams S_{V11} and S_{V12} for providing heat to at least one heat-consuming unit in the water-efficient process for hydrolytically depolymerizing a polyamide prepared from ε-caprolactam. More preferably, the process further comprises recycling at least a part of at least one of aqueous vapor streams S_{V11} and S_{V12} into step (i.2) as a component of the aqueous liquid stream S_{W}, wherein recycling at least a part of at least one of aqueous vapor streams S_{V11} and S_{V12} more preferably comprises condensing at least a part of at least one of streams S_{V11} and S_{V12} and the process comprises using at least a part of at least one of the aqueous vapor streams S_{V11} and S_{V12} for providing heat to at least one heat-consuming unit in the water-efficient process for hydrolytically depolymerizing a polyamide prepared from ε-caprolactam.

Preferably, (ii.1) comprises
(ii.1.1) feeding the aqueous liquid stream S_{WC} as a feed stream to a first sub-evaporation unit, EU11, obtaining an aqueous vapor stream S_{V11}, and an aqueous liquid stream S_{L11} comprising ε-caprolactam dissolved in water, wherein prior to feeding to EU11, the aqueous liquid stream S_{WC} is optionally passed through at least one solid-liquid separation unit F1;
(ii.1.2) feeding the aqueous liquid stream S_{L11} as a feed stream to a second sub-evaporation unit, EU12, obtaining an aqueous vapor stream S_{V12} and the aqueous liquid stream S_{L1} comprising ε-caprolactam dissolved in water, wherein prior to feeding to EU12, the aqueous liquid stream S_{L11} is optionally passed through at least one solid-liquid separation unit F2;
wherein (ii.1) comprises at least one of passing S_{WC} through F1 and passing S_{L11} through F2, wherein (ii.1) more preferably comprises passing S_{WC} through F1 and passing S_{L11} through F2. This is in particular illustrated in FIG. 3.

Preferably, at least one of the solid-liquid separation unit F1 and the solid-liquid separation unit F2, more preferably the solid-liquid separation unit F1 and the solid-liquid separation unit F2 are filtration units, wherein F1 more preferably has a mesh size in the range of from 0.5 to 5 mm, more preferably in the range of from 1 to 3 mm, and F2 more preferably has a mesh size in the range of from 0.5 to 5 mm, more preferably in the range of from 1 to 3 mm.

In the context of the present invention, preferably, the aqueous liquid stream which is fed to evaporation in at least two, more preferably in two evaporation units EU2 and EU3, more preferably in two serially coupled evaporation units EU2 and EU3 according to (ii.3), has a temperature in the range of from 75 to 105° C., more preferably in the range of from 85 to 105° C., more preferably in the range of from 95 to 105° C., more preferably at a pressure in the range of from 0.9 to 1.1 bar(abs), more preferably in the range of from 0.95 to 1.05 bar(abs).

Preferably from 85 to 100 weight-%, more preferably from 95 to 100 weight-%, of the aqueous liquid stream which is fed to evaporation in at least two, more preferably in two evaporation units EU2 and EU3, more preferably in two serially coupled evaporation units EU2 and EU3 according to (ii.3), consist of water and ε-caprolactam dissolved therein, wherein c_{CPL}, the concentration of ε-caprolactam, in said stream is in the range of from 7.5 to 17.5 weight-%, more preferably in the range of from 10 to 15 weight-%.

Preferably, (ii.3) comprises
(ii.3.1) feeding the aqueous liquid stream S_{L1} or the aqueous liquid stream S_{SLU}, more preferably the aqueous liquid stream S_{SLU}, to evaporation in a first evaporation unit EU2, obtaining an aqueous vapor stream S_{V2} and an aqueous liquid stream S_{L21}, wherein the concentration of ε-caprolactam in the stream S_{L21} is c_{CPLL21} with c_{CPLL21}>c_{CPL}, and wherein the concentration of water in the stream S_{V2} is c_{H2OV2} with c_{H2OV2}>c_{H2O};
(ii.3.2) feeding at least a part of the aqueous liquid stream S_{L21} to evaporation in a second evaporation unit EU3, obtaining an aqueous vapor stream S_{V3} and an aqueous liquid stream S_{L31}, wherein the concentration of ε-caprolactam in the stream S_{L31} is c_{CPLL31} with c_{CPLL31}>c_{CPLL21}, and wherein the concentration of water in the stream S_{V3} is c_{H2OV3} with c_{H2OV3}>c_{H2OL21}.

Preferably, the evaporation unit EU2 comprises, more preferably consists of, a film evaporator, more preferably a falling film evaporator, wherein said film evaporator is more preferably equipped with heating means to provide heat for evaporation.

Preferably, the evaporation unit EU3 comprises, more preferably consists of, a film evaporator, more preferably a falling film evaporator, wherein said film evaporator is more preferably equipped with heating means to provide heat for evaporation.

Preferably, the process comprises passing at least a part of at least one aqueous vapor stream S_{V}, preferably S_{V1}, more preferably at least a part of at least one of the aqueous vapor streams S_{V11} and S_{V12} through the heating means of the film evaporator comprised in EU3.

More preferably, the process comprises passing a part of S_{V11} through the heating means of the film evaporator comprised in EU3. This is in particular illustrated in FIG. 4. Alternatively, more preferably, the process comprises passing a part of S_{V12} through the heating means of the film evaporator comprised in EU3.

Preferably, (ii.3) further comprises recycling at least a part of the stream S_{V3} obtained from EU3 according to (ii.3.2) as feed stream, more preferably as vapor feed stream, into EU2 according to (ii.3.1). This is in particular illustrated in FIG. 5.

Preferably, the concentration of ε-caprolactam in the stream S_{L31}, c_{CPLL31}, is at least 40 weight-%, more preferably in the range of from 40 to 80 weight-%, more preferably in the range of from 50 to 75 weight-%, more preferably in the range of from 60 to 70 weight-%; and preferably from 65 to 100 weight-%, more preferably from 75 to 100 weight-%, more preferably from 85 to 100 weight-% of the stream S_{L31} consist of water and ε-caprolactam.

Preferably, (ii.3.2) comprises feeding at least a part of the aqueous liquid stream S_{L21} to evaporation in a second evaporation unit EU3, obtaining an aqueous vapor stream S_{V3}, and obtaining an aqueous liquid stream S_{L31} and a liquid stream S_{L32}, wherein the concentration of ε-caprolactam in the stream S_{L31} is c_{CPLL31} With c_{CPLL31}>c_{CPLL21}, wherein the concentration of ε-caprolactam in the stream S_{L32} is c_{CPLL32} With c_{CPLL32}>c_{CPLL21}, more preferably with c_{CPLL31}=c_{CPLL32}, and the concentration of water in the stream S_{V3} is c_{H2OV2} with c_{H2OV2}>c_{H2OL21}; wherein the stream S_{L32} is recycled as feed stream into EU3.

Preferably, the concentration of ε-caprolactam in the stream S_{L32}, c_{CPLL32}, is at least 40 weight-%, more preferably in the range of from 40 to 80 weight-%, more preferably in the range of from 50 to 75 weight-%, more preferably in the range of from 60 to 70 weight-%.

Preferably, feeding at least the part of the aqueous liquid stream S_{L21} to evaporation in the second evaporation unit EU3 according to (ii.3.2) comprises admixing the stream S_{L21} with the stream S_{L32} and feeding the combined stream to evaporation in the second evaporation unit EU3. This is in particular illustrated in FIG. 6.

Preferably, the process further comprises dividing the aqueous liquid stream S_{L21} into two aqueous liquid streams S_{L211} and S_{L212}, S_{L211} and S_{L212} having the chemical composition of S_{L21}, wherein S_{L211} is fed to evaporation unit EU3 according to (ii.3.2) and wherein S_{L212} is recycled as feed stream into EU2. This is in particular illustrated in FIG. 7.

Preferably, according to (ii.3), obtaining the aqueous vapor stream S_{V2} from EU2 comprises
(a) removing an aqueous stream S_{VL2} from EU2, S_{VL2} comprising an aqueous liquid phase and an aqueous vapor phase;
(b) subjecting the aqueous stream S_{VL2} to vapor-liquid separation, obtaining the aqueous vapor stream S_{V2}, and obtaining an aqueous liquid stream S_{L22}. This is in particular illustrated in FIG. 8.

Preferably, the process further comprises dividing the aqueous vapor stream S_{V2} obtained according to (ii.3)(b) into two aqueous vapor streams S_{V21} and S_{V22}, S_{V21} and S_{V22} having the chemical composition of S_{V2}, wherein S_{V21} is subjected to condensation in a condensation unit, wherein at least a part of the condensed stream S_{V21} is recycled into step (i.2) as a component of the aqueous liquid stream S_{W}. This is in particular illustrated in FIG. 9.

Preferably, the process comprises using the aqueous stream S_{V22} for providing heat to at least one heat-consuming unit in the water-efficient process for hydrolytically depolymerizing a polyamide prepared from ε-caprolactam.

Preferably, the process comprises passing the aqueous vapor stream S_{V22} through the heating means of the film evaporator comprised in EU2, obtaining a condensed stream S_{V22}, wherein at least a part of the condensed stream S_{V22} is recycled into step (i.2) as a component of the aqueous liquid stream S_{W}. This is in particular illustrated in FIG. 10.

Preferably, the process further comprises feeding the aqueous liquid stream S_{L22} obtained according to (ii.3)(b), to evaporation in the second evaporation unit EU3, optionally after admixing with the aqueous liquid stream S_{L21}. This is in particular illustrated in FIG. 11.

Preferably, the process comprises
(ii.2) feeding the aqueous liquid stream S_{WC} or the aqueous liquid stream S_{L1}, preferably the aqueous liquid stream S_{L1}, to a solid-liquid separation unit SLU, obtaining an aqueous liquid stream S_{SLU} comprising ε-caprolactam dissolved in water;
(ii.3) feeding the liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.

More preferably, the process according to the particular aspect disclosed in the foregoing comprises
(ii.2) feeding the aqueous liquid stream S_{WC} or the aqueous liquid stream S_{L1}, preferably the aqueous liquid stream S_{L1}, to a solid-liquid separation unit SLU, obtaining an aqueous liquid stream S_{SLU} comprising ε-caprolactam dissolved in water;
(ii.3) feeding the liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.

Preferably, the solid-liquid separation unit SLU comprises, more preferably consists of, one or more of a centrifuge, a decanter, a decanter centrifuge, and a filter, more preferably one or more of a decanter and a decanter centrifuge.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 2 to 4”, every embodiment in this range is meant to be explicitly disclosed for the skilled person, i.e. the wording of this term is to be understood by the skilled person as being synonymous to “The process of any one of embodiments 2, 3 and 4”. Further, it is explicitly noted that the following set of embodiments represents a suitably structured part of the general description directed to preferred aspects of the present invention, and, thus, suitably supports, but does not represent the claims of the present invention.
1. A water-efficient process for hydrolytically depolymerizing a polyamide prepared from ε-caprolactam, said polyamide being comprised in a solid material M, the process comprising
   (i) preparing an aqueous liquid stream S_{WC} containing ε-caprolactam dissolved in water, comprising
      (i.1) providing the solid material M containing the polyamide;
      (i.2) providing an aqueous liquid stream S_{W};
      (i.3) preparing an aqueous mixture of the solid material M provided according to (i.1) and the aqueous liquid stream S_{W} provided according to (i.2);
      (i.4) subjecting the aqueous mixture prepared according to (i.3) to depolymerization conditions in a chemical reactor unit R_{U}, obtaining the aqueous liquid stream S_{WC} containing ε-caprolactam dissolved in water;
   (ii) separating water from the aqueous liquid stream S_{WC} by evaporation in at least two evaporation units, obtaining at least one aqueous vapor stream S_{V}, wherein at least a part of at least one aqueous vapor stream S_{V} is recycled into step (i.2) as a component of the aqueous liquid stream S_{W}.
2. The process of embodiment 1, wherein preparing an aqueous liquid stream S_{WC} containing ε-caprolactam dissolved in water according to (i) comprises
   (i.1) providing the solid material M containing the polyamide, M having a temperature T_{M}, wherein T_{M}<T_{P},T_{P} being the melting point of the polyamide;
   (i.2) providing the aqueous liquid stream S_{W}, wherein from 50 weight-% to 100 weight-% of S_{W} consist of water and wherein S_{W} has a temperature T_{SW}, wherein T_{SW}>T_{P};
   (i.3) preparing an aqueous mixture of the solid material M provided according to (i.1) and the aqueous liquid stream S_{W} provided according to (i.2), comprising feeding the solid material M provided according to (i.1) and the liquid aqueous stream S_{W} provided according to (i.2) into a chemical reactor unit R_{U}, obtaining said mixture;
   (i.4) subjecting the aqueous liquid mixture prepared according to (i.3) to depolymerization conditions in the chemical reactor unit R_{U}, obtaining the aqueous liquid stream S_{WC} containing ε-caprolactam dissolved in water, wherein the depolymerization conditions comprise a depolymerization temperature T_{D} at a depolymerization pressure p_{D}, wherein T_{M}<T_{D}<T_{SW}.
3. The process of embodiment 2, wherein T_{D} is in the range of from 230 to 320° C., preferably in the range of from 250 to 300° C., more preferably in the range of from 270 to 295° C.
4. The process of embodiment 2 or 3, wherein T_{SW} is in the range of from 250 to 350° C., preferably in the range of from 260 to 330° C., more preferably in the range of from 290 to 325° C.
5. The process of any one of embodiments 2 to 4, wherein ΔT=T_{SW}−T_{P} and ΔT is in the range of from 10 to 70° C., preferably in the range of from 10 to 50° C., more preferably in the range of from 10 to 30° C.
6. The process of any one of embodiments 2 to 5, wherein p_{D} is in the range of from 40 to 120 bar, preferably in the range of from 50 to 100 bar, more preferably in the range of from 60 to 90 bar.
7. The process of any one of embodiments 1 to 6, preferably of any one of embodiments 2 to 6, wherein in the mixture prepared according to (i.3), at least 75 weight-%, preferably from 75 to 100 weight-%, more preferably from 85 to 100 weight-%, more preferably from 95 to 100 weight-% of the polyamide comprised in the solid material M are comprised in liquid form.
8. The process of any one of embodiments 1 to 7, wherein from 91 to 100 weight-%, preferably from 92 to 100 weight-%, more preferably from 95 to 100 weight-% of S_{W} provided according to (i.2) consist of water.
9. The process of any one of embodiments 1 to 8, wherein the solid material M provided according to (i.1) comprises, preferably consists of waste material, wherein said waste material preferably comprises textile waste material.
10. The process of any one of embodiments 1 to 9, wherein (ii) comprises
   (ii.1) optionally feeding the aqueous liquid stream S_{WC} as a feed stream to a first evaporation unit EU1, obtaining at least one aqueous vapor stream S_{V1}, and an aqueous liquid stream S_{L1} comprising ε-caprolactam dissolved in water;
   (ii.2) optionally feeding the aqueous liquid stream S_{WC} or the aqueous liquid stream S_{L1} to a solid-liquid separation unit, SLU, obtaining an aqueous liquid stream S_{SLU} comprising ε-caprolactam dissolved in water;
   (ii.3) feeding the aqueous liquid stream S_{WC} or the aqueous liquid stream S_{L1} or the aqueous liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained;
   wherein preferably from 75 to 100 weight-% of the aqueous liquid stream which is fed to evaporation according to (ii.3) consist of water and ε-caprolactam, said stream exhibiting a water concentration c_{H2O} and preferably having a concentration of ε-caprolactam c_{CPL} in the range of from 5 to 20 weight-%;
   the process further comprising recycling at least a part of at least one of streams S_{V2} and S_{V3} into step (i.2) as a component of the aqueous liquid stream S_{W}, said recycling preferably comprising condensing the at least one of streams S_{V2} and S_{V3}.
11. The process of embodiment 10, wherein (ii) comprises
   (ii.1) feeding the aqueous liquid stream S_{WC} as a feed stream to a first evaporation unit, EU1, obtaining at least one aqueous vapor stream S_{V1}, and an aqueous liquid stream S_{L1} comprising ε-caprolactam dissolved in water;
   (ii.2) optionally feeding the aqueous liquid stream S_{L1} to a solid-liquid separation unit SLU, obtaining an aqueous liquid stream S_{SLU} comprising ε-caprolactam dissolved in water;
   (ii.3) feeding the aqueous liquid stream S_{L1} or the aqueous liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.
12. The process of embodiment 11, preferably insofar as embodiment 11 is dependent on embodiment 6, wherein according to (ii.1), the aqueous liquid stream S_{WC} is fed to evaporation by depressurization in EU1, obtaining the at least one aqueous vapor stream S_{V1} and the aqueous liquid stream S_{L1}.
13. The process of embodiment 11 or 12, further comprising recycling at least a part of at least one aqueous vapor stream S_{V1} into step (i.2) as a component of the aqueous liquid stream S_{W}, wherein recycling at least a part of at least one stream S_{V1} into step (i.2) preferably comprises condensing at least a part of at least one stream S_{V1}.
14. The process of any one of embodiments 11 to 13, preferably of embodiment 13, comprising using at least a part of at least one aqueous vapor stream S_{V1} for providing heat to at least one heat-consuming unit in the water-efficient process for hydrolytically depolymerizing a polyamide prepared from ε-caprolactam.
15. The process of any one of embodiments 11 to 14, wherein (ii.1) comprises
   (ii.1.1) feeding the aqueous liquid stream S_{WC} as a feed stream to a first evaporation sub-unit EU11, obtaining an aqueous vapor stream S_{V11}, and an aqueous liquid stream S_{L11} comprising ε-caprolactam dissolved in water;
   (ii1.2) feeding the aqueous liquid stream S_{L11} as a feed stream to a second evaporation sub-unit EU12, obtaining an aqueous vapor stream S_{V12}, and the aqueous liquid stream S_{L1} comprising ε-caprolactam dissolved in water.
16. The process of embodiment 15, preferably insofar as embodiment 15 is dependent on embodiment 6, wherein according to (ii.1.1), the aqueous liquid stream S_{WC} is fed to evaporation by depressurization in EU11, obtaining the at least one aqueous vapor stream S_{V11} and the aqueous liquid stream S_{L11}, and wherein the aqueous liquid stream S_{L11} is fed to evaporation by depressurization in EU12, obtaining the at least one aqueous vapor stream S_{V12} and the aqueous liquid stream S_{L1}.
17. The process of embodiment 15 or 16, further comprising recycling at least a part of at least one of aqueous vapor streams S_{V11} and S_{V12} into step (i.2) as a component of the aqueous liquid stream S_{W}, wherein recycling at least a part of at least one of aqueous vapor streams S_{V11} and S_{V12} preferably comprises condensing at least a part of at least one of streams S_{V11} and S_{V12}.
18. The process of any one of embodiments 15 to 17, preferably of embodiment 17, comprising using at least a part of at least one of the aqueous vapor streams S_{V11} and S_{V12} for providing heat to at least one heat-consuming unit in the water-efficient process for hydrolytically depolymerizing a polyamide prepared from ε-caprolactam.
19. The process of any one of embodiments 15 to 18, wherein (ii.1) comprises
   (ii.1.1) feeding the aqueous liquid stream S_{WC} as a feed stream to a first sub-evaporation unit, EU11, obtaining an aqueous vapor stream S_{V11}, and an aqueous liquid stream S_{L11} comprising ε-caprolactam dissolved in water, wherein prior to feeding to EU11, the aqueous liquid stream S_{WC} is optionally passed through at least one solid-liquid separation unit F1;
   (ii.1.2) feeding the aqueous liquid stream S_{L11} as a feed stream to a second sub-evaporation unit, EU12, obtaining an aqueous vapor stream S_{V12} and the aqueous liquid stream S_{L1} comprising ε-caprolactam dissolved in water, wherein prior to feeding to EU12, the aqueous liquid stream S_{L11} is optionally passed through at least one solid-liquid separation unit F2;
   wherein (ii.1) comprises at least one of passing S_{WC} through F1 and passing S_{L11} through F2, wherein (ii.1) preferably comprises passing S_{WC} through F1 and passing S_{L11} through F2.
20. The process of embodiment 19, wherein at least one of the solid-liquid separation unit F1 and the solid-liquid separation unit F2, preferably the solid-liquid separation unit F1 and the solid-liquid separation unit F2 are filtration units, wherein F1 preferably has a mesh size in the range of from 0.5 to 5 mm, more preferably in the range of from 1 to 3 mm, and F2 preferably has a mesh size in the range of from 0.5 to 5 mm, more preferably in the range of from 1 to 3 mm.
21. The process of any one of embodiments 10 to 20, wherein the aqueous liquid stream which is fed to evaporation in at least two, preferably in two evaporation units EU2 and EU3, more preferably in two serially coupled evaporation units EU2 and EU3 according to (ii.3) has a temperature in the range of from 75 to 105° C., preferably in the range of from 85 to 105° C., more preferably in the range of from 95 to 105° C., preferably at a pressure in the range of from 0.9 to 1.1 bar(abs), more preferably in the range of from 0.95 to 1.05 bar(abs).
22. The process of any one of embodiments 10 to 21, wherein from 85 to 100 weight-%, more preferably from 95 to 100 weight-% of the aqueous liquid stream which is fed to evaporation in at least two, preferably in two evaporation units EU2 and EU3, more preferably in two serially coupled evaporation units EU2 and EU3 according to (ii.3) consist of water and ε-caprolactam dissolved therein, wherein c_{CPL}, the concentration of ε-caprolactam, in said stream is in the range of from 7.5 to 17.5 weight-%, preferably in the range of from 10 to 15 weight-%.
23. The process of any one of embodiments 10 to 22, wherein (ii.3) comprises
   (ii.3.1) feeding the aqueous liquid stream S_{L1} or the aqueous liquid stream S_{SLU}, preferably the aqueous liquid stream S_{SLU}, to evaporation in a first evaporation unit EU2, obtaining an aqueous vapor stream S_{V2} and an aqueous liquid stream S_{L21}, wherein the concentration of ε-caprolactam in the stream S_{L21} is c_{CPLL21} With c_{CPLL21}>c_{CPL}, and wherein the concentration of water in the stream S_{V2} is c_{H2OV2} with c_{H2OV2}>c_{H2O};
   (ii.3.2) feeding at least a part of the aqueous liquid stream S_{L21} to evaporation in a second evaporation unit EU3, obtaining an aqueous vapor stream S_{V3} and an aqueous liquid stream S_{L31}, wherein the concentration of ε-caprolactam in the stream S_{L31} is c_{CPLL31} with c_{CPLL31}>c_{CPLL21}, and wherein the concentration of water in the stream S_{V3} is c_{H2OV2} With c_{H2OV2}>c_{H2OL21}.
24. The process of embodiment 23, wherein the evaporation unit EU2 comprises, preferably consists of, a film evaporator, preferably a falling film evaporator, wherein said film evaporator is preferably equipped with heating means to provide heat for evaporation.
25. The process of embodiment 23 or 24, wherein the evaporation unit EU3 comprises, preferably consists of, a film evaporator, preferably a falling film evaporator, wherein said film evaporator is preferably equipped with heating means to provide heat for evaporation.
26. The process of embodiment 25, comprising passing at least a part of at least one aqueous vapor stream S_{V}, preferably S_{V1}, more preferably at least a part of at least one of the aqueous vapor streams S_{V11} and S_{V12} through the heating means of the film evaporator comprised in EU3.
27. The process of any one of embodiments 23 to 26, wherein (ii.3) further comprises recycling at least a part of the stream S_{V3} obtained from EU3 according to (ii.3.2) as feed stream, preferably as vapor feed stream, into EU2 according to (ii.3.1).
28. The process of embodiment of any one of embodiments 23 to 27, wherein the concentration of ε-caprolactam in the stream S_{L31}, c_{CPLL31}, is at least 40 weight-%, preferably in the range of from 40 to 80 weight-%, more preferably in the range of from 50 to 75 weight-%, more preferably in the range of from 60 to 70 weight-%, and wherein preferably 65 to 100 weight-%, more preferably from 75 to 100 weight-%, more preferably from 85 to 100 weight-% of the stream S_{L31} consist of water and ε-caprolactam.
29. The process of any one of embodiments 23 to 28, wherein (ii.3.2) comprises feeding at least a part of the aqueous liquid stream S_{L21} to evaporation in a second evaporation unit EU3, obtaining an aqueous vapor stream S_{V3}, and obtaining an aqueous liquid stream S_{L31} and a liquid stream S_{L32}, wherein the concentration of ε-caprolactam in the stream S_{L31} is c_{CPLL31} with c_{CPLL31}>c_{PLL21}, wherein the concentration of ε-caprolactam in the stream S_{L32} is c_{CPLL32} with c_{CPLL32}>c_{CPLL21}, preferably with c_{CPLL31}=c_{CPLL32}, and wherein the concentration of water in the stream S_{V3} is c_{H2OV2} with c_{H2OV2}>c_{H2OL21}; wherein the stream S_{L32} is recycled as feed stream into EU3.
30. The process of embodiment 29, wherein the concentration of ε-caprolactam in the stream S_{L32}, c_{CPLL32}, is at least 40 weight-%, preferably in the range of from 40 to 80 weight-%, more preferably in the range of from 50 to 75 weight-%, more preferably in the range of from 60 to 70 weight-%.
31. The process of embodiment 29 or 30, wherein feeding at least the part of the aqueous liquid stream S_{L21} to evaporation in the second evaporation unit EU3 according to (ii.3.2) comprises admixing the stream S_{L21} with the stream S_{L32} and feeding the combined stream to evaporation in the second evaporation unit EU3.
32. The process of any one of embodiments 23 to 31, further comprising dividing the aqueous liquid stream S_{L21} into two aqueous liquid streams S_{L211} and S_{L212}, S_{L211} and S_{L212} having the chemical composition of S_{L21}, wherein S_{L211} is fed to evaporation unit EU3 according to (ii.3.2) and wherein S_{L212} is recycled as feed stream into EU2.
33. The process of any one of embodiments 10 to 32, wherein according to (ii.3), obtaining the aqueous vapor stream S_{V2} from EU2 comprises
   (a) removing an aqueous stream S_{VL2} from EU2, S_{VL2} comprising an aqueous liquid phase and an aqueous vapor phase;
   (b) subjecting the aqueous stream S_{VL2} to vapor-liquid separation, obtaining the aqueous vapor stream S_{V2}, and obtaining an aqueous liquid stream S_{L22}.
34. The process of embodiment 33, further comprising dividing the aqueous vapor stream S_{V2} into two aqueous vapor streams S_{V21} and S_{V22}, S_{V21} and S_{V22} having the chemical composition of S_{V2}, wherein S_{V21} is subjected to condensation in a condensation unit, wherein at least a part of the condensed stream S_{V21} is recycled into step (i.2) as a component of the aqueous liquid stream S_{W}.
35. The process of embodiment 34, comprising using the aqueous stream S_{V22} for providing heat to at least one heat-consuming unit in the water-efficient process for hydrolytically depolymerizing a polyamide prepared from ε-caprolactam.
36. The process of embodiment 35, insofar as embodiment 34 is dependent on embodiment 24, the process comprising passing the aqueous vapor stream S_{V22} through the heating means of the film evaporator comprised in EU2, obtaining a condensed stream S_{V22}, wherein at least a part of the condensed stream S_{V22} is recycled into step (i.2) as a component of the aqueous liquid stream S_{W}.
37. The process of any one of embodiments 33 to 36, further comprising feeding the aqueous liquid stream S_{L22} to evaporation in the second evaporation unit EU3, optionally after admixing with the aqueous liquid stream S_{L21}.
38. The process of any one of embodiments 10 to 37, preferably of any one of embodiments 11
   to 37, comprising (ii.2) feeding the aqueous liquid stream S_{WC} or the aqueous liquid stream S_{L1}, preferably the aqueous liquid stream S_{L1}, to a solid-liquid separation unit SLU, obtaining an aqueous liquid stream S_{SLU} comprising ε-caprolactam dissolved in water;
   (ii.3) feeding the liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.
39. The process of embodiment 38, wherein the solid-liquid separation unit SLU comprises, preferably consists of, one or more of a centrifuge, a decanter, a decanter centrifuge, and a filter, preferably one or more of a decanter and a decanter centrifuge.

In the context of the present invention, it is noted that the term “polyamide prepared from ε-caprolactam” as used herein refers to “polyamide 6” being characterized by the formula (—NH—(CH₂)₅—CO—)ₙ. The term “bar” as used in the context of the present invention refers to “bar(abs)”, i.e. bar (absolute), sometimes also referred to “bara”.

The term “textile material” covers textile raw materials and non-textile raw materials that are processed by various methods into linear, planar and spatial structures. It concerns the linear textile structures produced from them, such as yarns, twisted yarns and ropes, the sheet-like textile structures, such as woven fabrics, knitted fabrics, braids, stitch-bonded fabrics, nonwovens and felts, and the three-dimensional textile structures, i.e. body structures, such as textile hoses, stockings or textile semi-finished products; and it further concerns those finished products which, using the aforementioned products, are brought into a saleable condition by making up, opening up and/or other operations for onward transmission to the processor, the trade or the end consumer.

The term “textile waste material” covers a textile material as defined above, the inherent value of which has been consumed from the perspective of its current holder and, thus, is an end-of-life material for said holder.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

### EXAMPLE

47.2 kg of filtered (depolymerization) reactor discharge (aqueous discharge) were metered continuously into a first water removal distillation. The dosing rate was 7.5 kg/h. The concentration of caprolactam in the starting material (aqueous discharge) was about 3.6 to 5.8 wt.-%. The pressure in the apparatus was 1 bar(abs). The temperature in the sump tank was 103° C. The top temperature of the apparatus was 100.1° C. The ratio of reflux to condensate removal was 6:2. The apparatus comprised a sump tank of approx. 6 L, a column structure approx. 50 cm high including unstructured packing. The apparatus further comprised a head condenser including a collection tank, and a natural circulation heat exchanger which circulates a quantity of raw material removed from the sump tank around the sump tank in order to heat it up. The sump tank is a jacketed tank. The obtained product weighed 8.5 kg, with a caprolactam content of 15 wt.-%. 38.7 kg of condensate were formed via the top condenser of the apparatus. In a second, downstream dewatering stage, this product was then metered into a container where further water was distilled off (batchwise) at 90° C. and 200 mbar(abs) pressure. An approx. 0.4 m high batch column was located on the container, and a top condenser with reflux divider was connected to the upper part. A circuit with a pump and a heat exchanger for energy input was located on the container. This was operated at 110° C. After the 2^{nd} drainage, the product had a caprolactam content of 32.3 wt.-%. The residual water content of this product was determined to be 12 wt.-%.

### DESCRIPTION OF THE FIGURES

FIG. 1 is a schematic representation of a production unit used for the process according to preferred embodiments of the invention

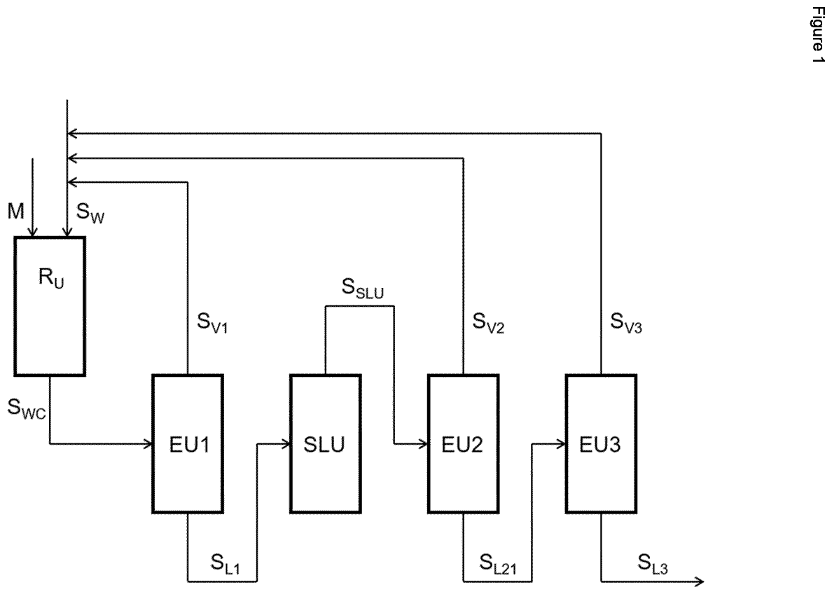

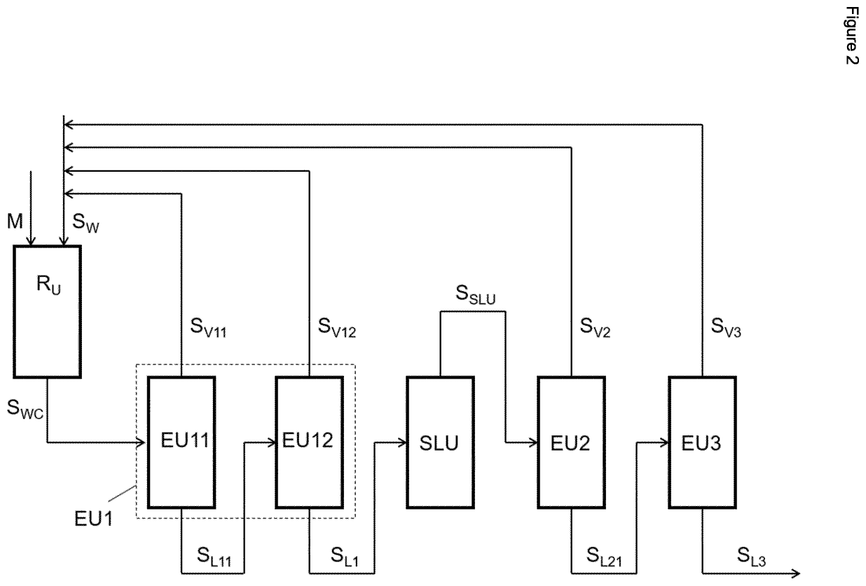

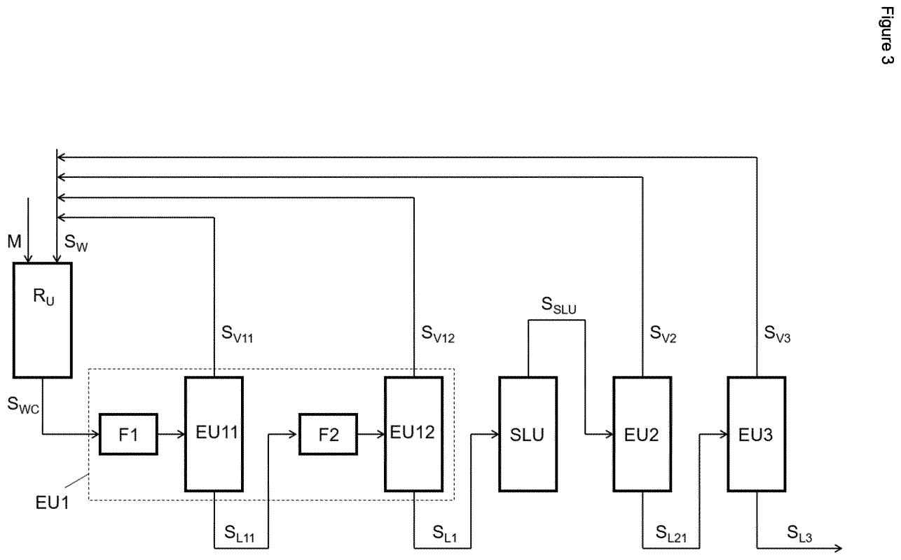

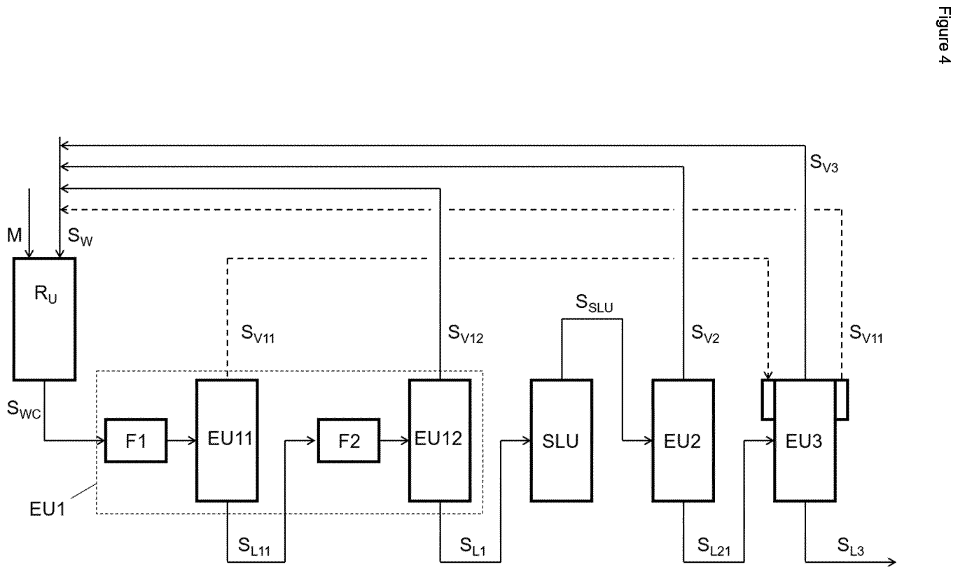

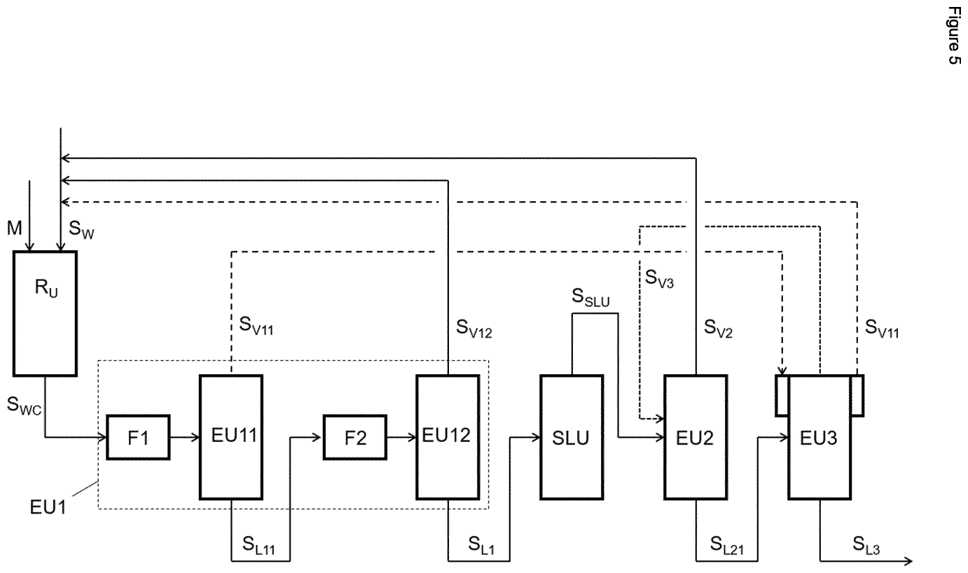

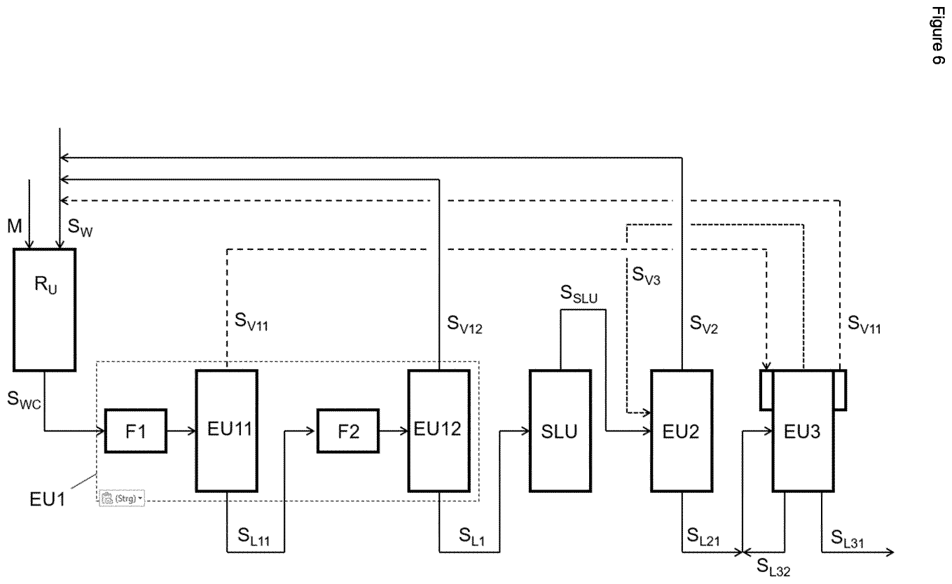

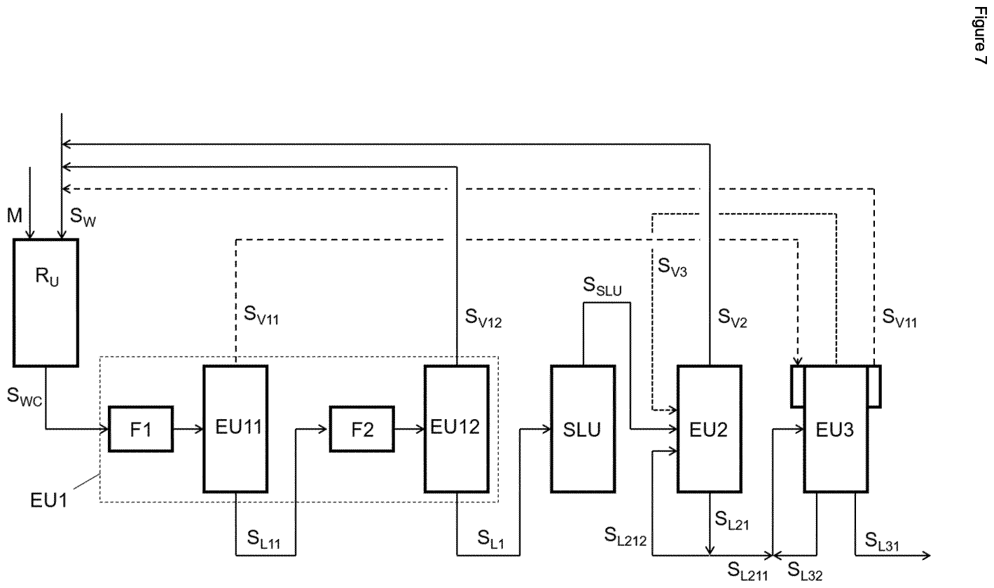

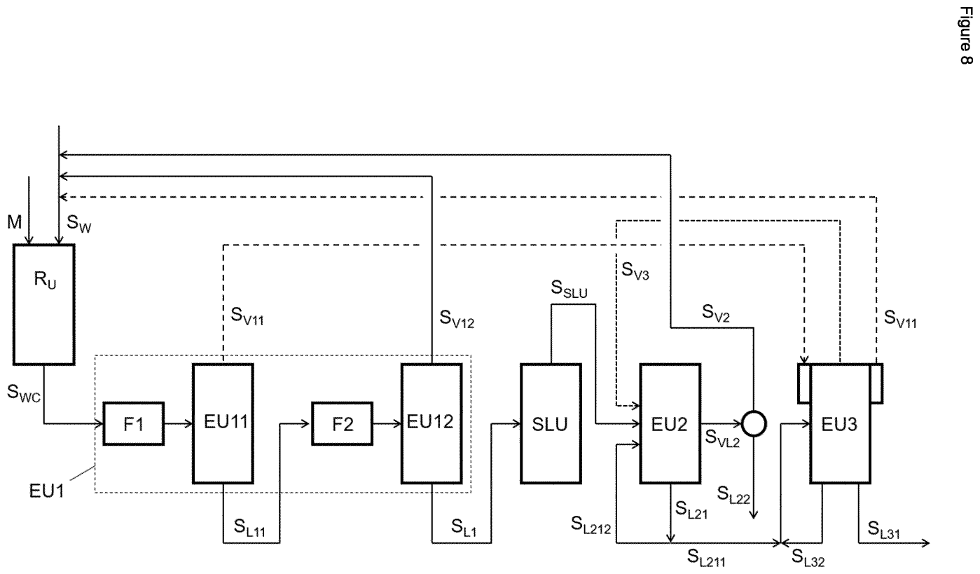

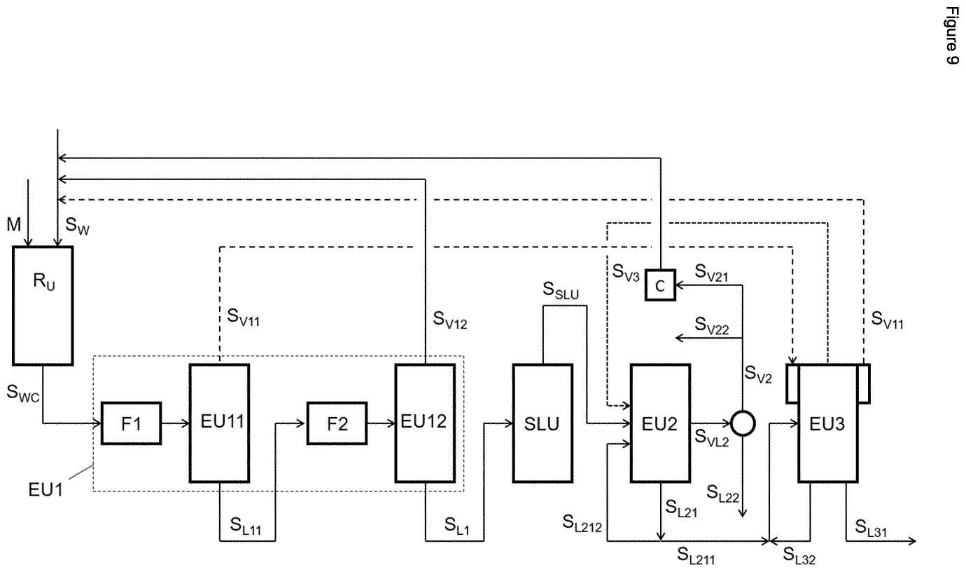

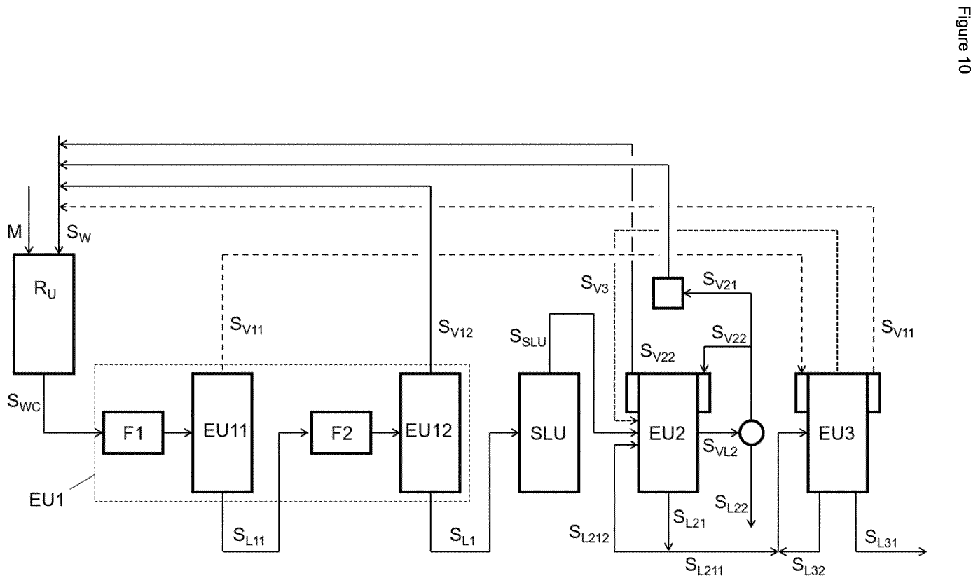

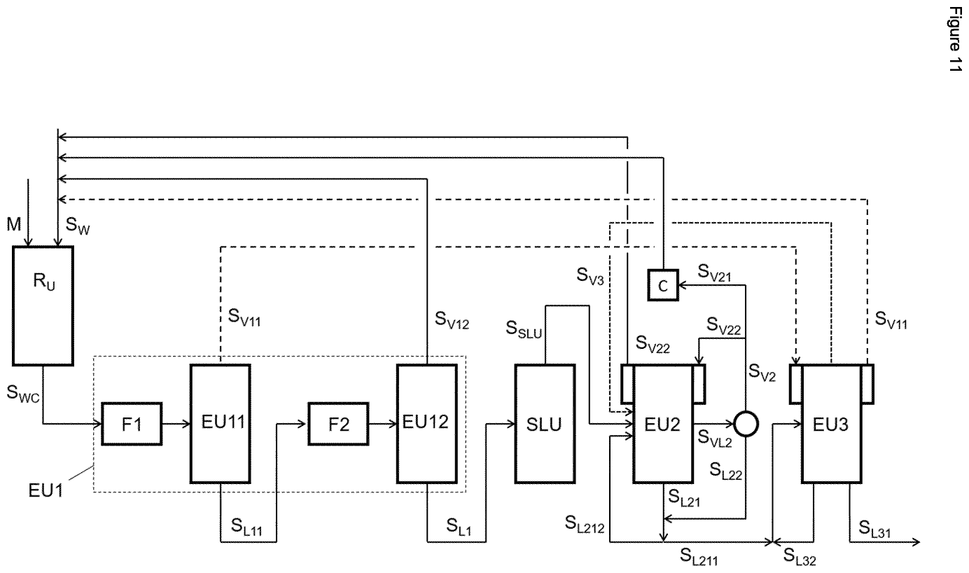

The production unit comprises a reactor unit R_{U}, three evaporation units EU1, EU2 and EU3 and a solid-liquid separation unit SLU. The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{WC} is removed from the bottom of R_{U}, S_{WC} comprising ε-caprolactam dissolved in water. The aqueous liquid stream S_{WC} is fed into the evaporation unit EU1 obtaining an aqueous vapor stream S_{V1}, and an aqueous liquid stream S_{L1} comprising ε-caprolactam dissolved in water. The aqueous vapor stream S_{V1} is recycled as a component of the aqueous liquid stream S_{W}, preferably via condensation. The aqueous liquid stream S_{L1} is passed through the solid-liquid separation unit SLU obtaining an aqueous liquid stream S_{SLU} comprising ε-caprolactam dissolved in water. The aqueous liquid stream S_{SLU} is then fed to evaporation in two evaporation units EU2 and EU3, said two units are serially coupled as shown in FIG. 1. An aqueous vapor stream S_{V2} is obtained from EU2 and an aqueous vapor stream S_{V3} is obtained from EU3. The aqueous vapor streams S_{V2} and S_{V3} are recycled as a component of the aqueous liquid stream S_{W}, preferably via condensation. Further, an aqueous liquid stream S_{L21} comprising ε-caprolactam dissolved in water is removed from EU2 and fed into EU3 and an aqueous liquid stream S_{L3} comprising ε-caprolactam dissolved in water is obtained and removed from EU3.

FIG. 2 is a schematic representation of a production unit used for the process according to preferred embodiments of the invention

The production unit comprises a reactor unit R_{U}, three evaporation units EU1, EU2 and EU3 and a solid-liquid separation unit SLU, except that compared to FIG. 1 the evaporation unit EU1 comprises an evaporation sub-unit EU11 and an evaporation sub-unit EU12. The aqueous liquid stream S_{WC} removed from the bottom of R_{U} is passed through the sub-unit EU11 obtaining an aqueous vapor stream S_{V11}, and an aqueous liquid stream S_{L11} comprising ε-caprolactam dissolved in water. The aqueous liquid stream S_{L11} is then fed into the second evaporation sub-unit EU12, as a feed stream, to obtain an aqueous vapor stream S_{V12}, and the aqueous liquid stream S_{L1} comprising ε-caprolactam dissolved in water. The aqueous vapor streams S_{V11} and S_{V12} are recycled as a component of the aqueous liquid stream S_{W}, preferably via condensation. Downstream of EU1, the process is carried out as the process in FIG. 1.

FIG. 3 is a schematic representation of a production unit used for the process according to preferred embodiments of the invention

The production unit comprises a reactor unit R_{U}, three evaporation units EU1, EU2 and EU3 and a solid-liquid separation unit SLU, except that compared to FIG. 2 the evaporation unit EU1 comprises in addition to evaporation sub-units EU11 and EU12, two solid-liquid separation units F1 and F2. Hence, the aqueous liquid stream S_{WC} removed from the bottom of R_{U} is passed through the solid-liquid separation unit F1, preferably filtration unit F1, wherein F1 preferably has a mesh size in the range of from 0.5 to 5 mm, more preferably in the range of from 1 to 3 mm, prior to being fed into the sub-unit EU11 to obtain an aqueous vapor stream S_{V11}, and an aqueous liquid stream S_{L11} comprising ε-caprolactam dissolved in water. The aqueous liquid stream S_{L11} is then passed through the solid-liquid separation unit F2, preferably filtration unit F2, wherein F2 preferably has a mesh size in the range of from 0.5 to 5 mm, more preferably in the range of from 1 to 3 mm, prior to being fed into the second evaporation sub-unit EU12, as a feed stream, to obtain an aqueous vapor stream S_{V12}, and the aqueous liquid stream S_{L1} comprising ε-caprolactam dissolved in water. The aqueous vapor streams S_{V11} and S_{V12} are recycled as a component of the aqueous liquid stream S_{W}, preferably via condensation. Downstream of EU1, the process is carried out as the process in FIG. 1 or 2.

FIG. 4 is a schematic representation of a production unit used for the process according to preferred embodiments of the invention

The production unit comprises a reactor unit R_{U}, three evaporation units EU1, EU2 and EU3 and a solid-liquid separation unit SLU, wherein the evaporation unit EU1 comprises sub-units EU11 and EU12, two solid-liquid separation units F1 and F2. Further, compared to FIG. 3, EU3 of the production unit comprises heating means to provide heat for evaporation. The aqueous liquid stream S_{WC} removed from the bottom of R_{U} is passed through the solid-liquid separation unit F1, preferably filtration unit F1, wherein F1 preferably has a mesh size in the range of from 0.5 to 5 mm, more preferably in the range of from 1 to 3 mm, prior to being fed into the sub-unit EU11 to obtain an aqueous vapor stream S_{V11}, and an aqueous liquid stream S_{L11} comprising ε-caprolactam dissolved in water. The aqueous liquid stream S_{L11} is then passed through the solid-liquid separation unit F2, preferably filtration unit F2, wherein F2 preferably has a mesh size in the range of from 0.5 to 5 mm, more preferably in the range of from 1 to 3 mm, prior to being fed into the second evaporation sub-unit EU12, as a feed stream, to obtain an aqueous vapor stream S_{V12}, and the aqueous liquid stream S_{L1} comprising ε-caprolactam dissolved in water. The aqueous vapor stream S_{V12} is recycled as a component of the aqueous liquid stream S_{W}, preferably via condensation, while the aqueous vapor stream S_{V11} is passed through the heating means of the film evaporator comprised in EU3. It is also conceivable that only a part of S_{V11} be passed through the heating means of the film evaporator comprised in EU3, the other part of S_{V11} being recycled as a component of the aqueous liquid stream S_{W} (not shown in FIG. 4). Downstream of EU1, the process is carried out as the process in FIG. 3 with the exception that EU3 comprises heating means.

FIG. 5 is a schematic representation of a production unit used for the process according to preferred embodiments of the invention

As in FIG. 4, the production unit comprises a reactor unit R_{U}, three evaporation units EU1, EU2 and EU3 and a solid-liquid separation unit SLU, wherein EU1 comprises sub-units EU11 and EU12, two solid-liquid separation units F1 and F2 and wherein EU3 comprises heating means to provide heat for evaporation. The process illustrated by FIG. 5 is carried out as the one illustrated by FIG. 4 except that the aqueous vapor stream S_{V3} is recycled as a feed stream into EU2.

FIG. 6 is a schematic representation of a production unit used for the process according to preferred embodiments of the invention

As in FIG. 5, the production unit comprises a reactor unit R_{U}, three evaporation units EU1, EU2 and EU3 and a solid-liquid separation unit SLU, wherein EU1 comprises sub-units EU11 and EU12, two solid-liquid separation units F1 and F2 and wherein EU3 comprises heating means to provide heat for evaporation. The process illustrated by FIG. 6 is run as the one illustrated by FIG. 5 except that an aqueous liquid stream S_{L31} and a liquid stream S_{L32}, wherein the concentration of ε-caprolactam in the stream S_{L31} is c_{CPLL31} With c_{CPLL31}>c_{CPLL21}, wherein the concentration of ε-caprolactam in the stream S_{L32} is c_{CPLL32} With c_{CPLL32}>c_{CPLL21}, preferably with c_{CPLL31}=c_{CPLL32}, and wherein the concentration of water in the stream S_{V3} is c_{H2OV2} with c_{H2OV2}>c_{H2OL21}, are removed from EU3. The liquid stream S_{L32} is recycled as feed stream into EU3. Further, the aqueous liquid stream S_{L21} removed from EU2 comprising ε-caprolactam dissolved in water is admixed with the liquid stream S_{L32}. The combined stream is then fed to evaporation into EU3.

FIG. 7 is a schematic representation of a production unit used for the process according to preferred embodiments of the invention

As in FIG. 6, the production unit comprises a reactor unit R_{U}, three evaporation units EU1, EU2 and EU3 and a solid-liquid separation unit SLU, wherein EU1 comprises sub-units EU11 and EU12, two solid-liquid separation units F1 and F2 and wherein EU3 comprises heating means to provide heat for evaporation. The process illustrated by FIG. 7 is carried out as the one illustrated by FIG. 6 except that the aqueous liquid stream S_{L21} removed from EU2 comprising ε-caprolactam dissolved in water is divided in two aqueous liquid streams S_{L211} and S_{L212}. S_{L211} and S_{L212} have the chemical composition of S_{L21}. The aqueous liquid stream S_{L212} is recycled as feed stream in EU2 and the aqueous liquid stream S_{L211} is admixed with the liquid stream S_{L32}. The combined stream is then fed to evaporation into EU3.

FIG. 8 is a schematic representation of a production unit used for the process according to preferred embodiments of the invention

As in FIG. 7, the production unit comprises a reactor unit R_{U}, three evaporation units EU1, EU2 and EU3 and a solid-liquid separation unit SLU, wherein EU1 comprises sub-units EU11 and EU12, two solid-liquid separation units F1 and F2 and wherein EU3 comprises heating means to provide heat for evaporation. However, in FIG. 8, the production unit further comprises a vapor-liquid separation unit (circle in FIG. 8) downstream of EU2. A stream S_{VL2}, comprising an aqueous liquid phase and an aqueous vapor phase, is removed from EU2 and passed through the vapor-liquid separation unit to vapor-liquid separation, obtaining the aqueous vapor stream S_{V2}, and obtaining an aqueous liquid stream S_{L22}. The aqueous vapor stream S_{V2} is recycled as a component of the aqueous liquid stream S_{W}. Apart from said differences, the process illustrated by FIG. 8 is carried out as the one illustrated by FIG. 7.

FIG. 9 is a schematic representation of a production unit used for the process according to preferred embodiments of the invention

As in FIG. 8, the production unit comprises a reactor unit R_{U}, three evaporation units EU1, EU2 and EU3, a solid-liquid separation unit SLU and a vapor-liquid separation unit downstream of EU2, wherein EU1 comprises sub-units EU11 and EU12, two solid-liquid separation units F1 and F2 and wherein EU3 comprises heating means to provide heat for evaporation. However, in FIG. 9, the production unit further comprises a condensation unit downstream of the vapor-liquid separation unit. The aqueous vapor stream S_{V2} is divided into two aqueous vapor streams S_{V21} and S_{V22}, S_{V21} and S_{V22} having the chemical composition of S_{V2}. The aqueous vapor stream S_{V21} is subjected to condensation in the condensation unit C. The condensed stream S_{V21} is recycled as a component of the aqueous liquid stream S_{W}. Apart from said differences, the process illustrated by FIG. 9 is carried out as the one illustrated by FIG. 8.

FIG. 10 is a schematic representation of a production unit used for the process according to preferred embodiments of the invention

As in FIG. 9, the production unit comprises a reactor unit R_{U}, three evaporation units EU1, EU2 and EU3, a solid-liquid separation unit SLU, a vapor-liquid separation unit downstream of EU2 and a condensation unit C, wherein EU1 comprises sub-units EU11 and EU12, two solid-liquid separation units F1 and F2 and wherein EU3 comprises heating means to provide heat for evaporation. The process illustrated by FIG. 10 is carried out as the one illustrated by FIG. 9 except that the aqueous vapor stream S_{V22} is used for providing heat to EU2. Said stream S_{V22} is passed through the heating means of EU2, film evaporator, to obtain a condensed stream S_{V22} which is then recycled as a component of the aqueous liquid stream S_{W}.

FIG. 11 is a schematic representation of a production unit used for the process according to preferred embodiments of the invention

As in FIG. 10, the production unit comprises a reactor unit R_{U}, three evaporation units EU1, EU2 and EU3, a solid-liquid separation unit SLU, a vapor-liquid separation unit downstream of EU2 and a condensation unit C, wherein EU1 comprises sub-units EU11 and EU12, two solid-liquid separation units F1 and F2 and wherein EU3 comprises heating means to provide heat for evaporation. The process illustrated by FIG. 11 is carried out as the one illustrated by FIG. 10 except that the aqueous liquid stream S_{L22} is admixed with the aqueous liquid stream S_{L21} and subjected to evaporation to EU3.

1.-17. (canceled)

18. A water-efficient process for hydrolytically depolymerizing a polyamide prepared from ε-caprolactam, said polyamide being comprised in a solid material M, the process comprising
(i) preparing an aqueous liquid stream S_{WC} containing ε-caprolactam dissolved in water, comprising
(i.1) providing the solid material M containing the polyamide;
(i.2) providing an aqueous liquid stream S_{W};
(i.3) preparing an aqueous mixture of the solid material M provided according to (i.1) and the aqueous liquid stream S_{W} provided according to (i.2);
(i.4) subjecting the aqueous mixture prepared according to (i.3) to depolymerization conditions in a chemical reactor unit R_{U}, obtaining the aqueous liquid stream S_{WC} containing ε-caprolactam dissolved in water;
(ii) separating water from the aqueous liquid stream S_{WC} by evaporation in at least two evaporation units, obtaining at least one aqueous vapor stream S_{V}, wherein at least a part of at least one aqueous vapor stream S_{V} is recycled into step (i.2) as a component of the aqueous liquid stream S_{W}.

19. The process of claim 18, wherein preparing an aqueous liquid stream S_{WC} containing ε-caprolactam dissolved in water according to (i) comprises
(i.1) providing the solid material M containing the polyamide, M having a temperature T_{M}, wherein T_{M}<T_{P}, T_{P} being the melting point of the polyamide;
(i.2) providing the aqueous liquid stream S_{W}, wherein from 50 weight-% to 100 weight-% of S_{W} consist of water and wherein S_{W} has a temperature T_{SW}, wherein T_{SW}>T_{P};
(i.3) preparing an aqueous mixture of the solid material M provided according to (i.1) and the aqueous liquid stream S_{W} provided according to (i.2), comprising feeding the solid material M provided according to (i.1) and the liquid aqueous stream S_{W} provided according to (i.2) into a chemical reactor unit R_{U}, obtaining said mixture;
(i.4) subjecting the aqueous liquid mixture prepared according to (i.3) to depolymerization conditions in the chemical reactor unit R_{U}, obtaining the aqueous liquid stream S_{WC} containing ε-caprolactam dissolved in water, wherein the depolymerization conditions comprise a depolymerization temperature T_{D} at a depolymerization pressure p_{D}, wherein T_{M}<T_{D}<T_{SW}.

20. The process of claim 18, wherein ΔT=T_{SW}−T_{P} and ΔT is in the range of from 10 to 70° C.

21. The process of claim 18, wherein from 91 to 100 weight-% of S_{W} provided according to (i.2) consist of water.

22. The process of claim 18, wherein (ii) comprises
(ii.1) optionally feeding the aqueous liquid stream S_{WC} as a feed stream to a first evaporation unit EU1, obtaining at least one aqueous vapor stream S_{V1}, and an aqueous liquid stream S_{L1} comprising ε-caprolactam dissolved in water;
(ii.2) optionally feeding the aqueous liquid stream S_{WC} or the aqueous liquid stream S_{L1} to a solid-liquid separation unit, SLU, obtaining an aqueous liquid stream S_{SLU} comprising ε-caprolactam dissolved in water;
(ii.3) feeding the aqueous liquid stream S_{WC} or the aqueous liquid stream S_{L1} or the aqueous liquid stream S_{SLU} to evaporation in at least two evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained;
wherein preferably from 75 to 100 weight-% of the aqueous liquid stream which is fed to evaporation according to (ii.3) consist of water and ε-caprolactam, said stream exhibiting a water concentration c_{H2O};
the process further comprising recycling at least a part of at least one of streams S_{V2} and S_{V3} into step (i.2) as a component of the aqueous liquid stream S_{W}.

23. The process of claim 22, wherein (ii) comprises
(ii.1) feeding the aqueous liquid stream S_{WC} as a feed stream to a first evaporation unit, EU1, obtaining at least one aqueous vapor stream S_{V1}, and an aqueous liquid stream S_{L1} comprising ε-caprolactam dissolved in water;
(ii.2) optionally feeding the aqueous liquid stream S_{L1} to a solid-liquid separation unit SLU, obtaining an aqueous liquid stream S_{SLU} comprising ε-caprolactam dissolved in water;
(ii.3) feeding the aqueous liquid stream S_{L1} or the aqueous liquid stream S_{SLU} to evaporation in at least two evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.

24. The process of claim 23, wherein (ii.1) comprises
(ii.1.1) feeding the aqueous liquid stream S_{WC} as a feed stream to a first evaporation sub-unit EU11, obtaining an aqueous vapor stream S_{V11}, and an aqueous liquid stream S_{L11} comprising ε-caprolactam dissolved in water;
(ii.1.2) feeding the aqueous liquid stream S_{L11} as a feed stream to a second evaporation sub-unit EU12, obtaining an aqueous vapor stream S_{V12}, and the aqueous liquid stream S_{L1} comprising ε-caprolactam dissolved in water.

25. The process of claim 24, wherein (ii.1) comprises
(ii.1.1) feeding the aqueous liquid stream S_{WC} as a feed stream to a first sub-evaporation unit, EU11, obtaining an aqueous vapor stream S_{V11}, and an aqueous liquid stream S_{L11} comprising ε-caprolactam dissolved in water, wherein prior to feeding to EU11, the aqueous liquid stream S_{WC} is optionally passed through at least one solid-liquid separation unit F1;
(ii.1.2) feeding the aqueous liquid stream S_{L11} as a feed stream to a second sub-evaporation unit, EU12, obtaining an aqueous vapor stream S_{V12} and the aqueous liquid stream S_{L1} comprising ε-caprolactam dissolved in water, wherein prior to feeding to EU12, the aqueous liquid stream S_{L11} is optionally passed through at least one solid-liquid separation unit F2;
wherein (ii.1) comprises at least one of passing S_{WC} through F1 and passing S_{L11} through F2,
wherein (ii.1) preferably comprises passing S_{WC} through F1 and passing S_{L11} through F2.

26. The process of claim 22, wherein (ii.3) comprises
(ii.3.1) feeding the aqueous liquid stream S_{L1} or the aqueous liquid stream S_{SLU}, to evaporation in a first evaporation unit EU2, obtaining an aqueous vapor stream S_{V2} and an aqueous liquid stream S_{L21}, wherein the concentration of ε-caprolactam in the stream S_{L21} is c_{CPLL21} with c_{CPLL21}>c_{CPL}, and wherein the concentration of water in the stream S_{V2} is c_{H2OV2} with c_{H2OV2}>c_{H2O};
(ii.3.2) feeding at least a part of the aqueous liquid stream S_{L21} to evaporation in a second evaporation unit EU3, obtaining an aqueous vapor stream S_{V3} and an aqueous liquid stream S_{L31}, wherein the concentration of ε-caprolactam in the stream S_{L31} is c_{CPLL31} with c_{CPLL31}>c_{CPLL21}, and wherein the concentration of water in the stream S_{V3} is c_{H2OV2} with c_{H2OV2}>c_{H2OL21}.

27. The process of claim 28, wherein the evaporation unit EU2 comprises a film evaporator.

28. The process of claim 27, the process comprising passing at least a part of at least one aqueous vapor stream S_{V} through the heating means of the film evaporator comprised in EU3.

29. The process of claim 26, wherein (ii.3.2) comprises feeding at least a part of the aqueous liquid stream S_{L21} to evaporation in a second evaporation unit EU3, obtaining an aqueous vapor stream S_{V3}, and obtaining an aqueous liquid stream S_{L31} and a liquid stream S_{L32}, wherein the concentration of ε-caprolactam in the stream S_{L31} is c_{CPLL31} with c_{CPLL31}>c_{CPLL21}, wherein the concentration of ε-caprolactam in the stream S_{L32} is c_{CPLL32} with c_{CPLL32}>c_{CPLL21}, and wherein the concentration of water in the stream S_{V3} is c_{H2OV2} with c_{H2OV2}>c_{H2OL21}; wherein the stream S_{L32} is recycled as feed stream into EU3.

30. The process of claim 29, wherein feeding at least the part of the aqueous liquid stream S_{L21} to evaporation in the second evaporation unit EU3 according to (ii.3.2) comprises admixing the stream S_{L21} with the stream S_{L32} and feeding the combined stream to evaporation in the second evaporation unit EU3.

31. The process of claim 22, wherein according to (ii.3), obtaining the aqueous vapor stream S_{V2} from EU2 comprises
(a) removing an aqueous stream S_{VL2} from EU2, S_{VL2} comprising an aqueous liquid phase and an aqueous vapor phase;
(b) subjecting the aqueous stream S_{VL2} to vapor-liquid separation, obtaining the aqueous vapor stream S_{V2}, and obtaining an aqueous liquid stream S_{L22}.

32. The process of claim 31, further comprising dividing the aqueous vapor stream S_{V2} into two aqueous vapor streams S_{V21} and S_{V22}, S_{V21} and S_{V22} having the chemical composition of S_{V2}, wherein S_{V21} is subjected to condensation in a condensation unit, wherein at least a part of the condensed stream S_{V21} is recycled into step (i.2) as a component of the aqueous liquid stream S_{W}.

33. The process of claim 31, further comprising feeding the aqueous liquid stream S_{L22} to evaporation in the second evaporation unit EU3, optionally after admixing with the aqueous liquid stream S_{L21}.

34. The process of claim 22, comprising
(ii.2) feeding the aqueous liquid stream S_{WC} or the aqueous liquid stream S_{L1}, preferably the aqueous liquid stream S_{L1}, to a solid-liquid separation unit SLU, obtaining an aqueous liquid stream S_{SLU} comprising ε-caprolactam dissolved in water;
(ii.3) feeding the liquid stream S_{SLU} to evaporation in at least two, preferably in two evaporation units EU2 and EU3, more preferably in two serially coupled evaporation units EU2 and EU3, wherein an aqueous vapor stream S_{V2} is obtained from EU2 and an aqueous vapor stream S_{V3} is obtained from EU3, and wherein from EU3, an aqueous liquid stream S_{L3} comprising ε-caprolactam dissolved in water is obtained.
